(19) United States
(12) Patent Application Publication
(10) Pub. No.: US 2024/0079112 A1
(43) Pub. Date: Mar. 7, 2024

(54) PHYSIOLOGICAL PREDICTIONS USING MACHINE LEARNING

(71) Applicant: Apple Inc., Cupertino, CA (US)

(72) Inventors: Andrew MILLER, Brooklyn, NY (US); Gregory DARNELL, Palo Alto, CA (US); Guillermo R. SAPIRO, Durham, NC (US); Seyedeh Sana TONEKABONI, North York (CA); You REN, Mercer Island, WA (US); Achille NAZARET, New York City, NY (US)

(21) Appl. No.: 18/076,367

(22) Filed: Dec. 6, 2022

Related U.S. Application Data
(60) Provisional application No. 63/404,531, filed on Sep. 7, 2022.
(60) Provisional application No. 63/407,602, filed on Sep. 16, 2022.

(51) Int. Cl. G16H 20/30

(52) U.S. Cl. CPC G16H 20/30

(57) ABSTRACT

The subject technology provides a framework for generating physiological predictions for a user of an electronic device. The physiological predictions may include user-specific predictions of a heartrate, a heartrate range, a number of steps, a number of calories, or other physiological conditions or aspects that may occur if the user engages in a future activity, such as a future workout. The physiological predictions may be generated by a machine learning model that incorporates a physiological state equation, and that generates, and utilizes, a user-specific embedding, along with user-agnostic parameters of the future activity, to make the predictions.

### CROSS REFERENCE TO RELATED APPLICATIONS

This application claims the benefit of priority to U.S. Provisional Patent Application No. 63/404,531, entitled, “Physiological Predictions Using Machine Learning”, filed on Sep. 7, 2022, and U.S. Provisional Patent Application No. 63/407,602, entitled, “Physiological Predictions Using Machine Learning”, filed on Sep. 16, 2022, the disclosure of each of which is hereby incorporated herein in its entirety.

### TECHNICAL FIELD

The present description generally relates to developing machine learning applications.

### BACKGROUND

Software engineers and scientists have been using computer hardware for machine learning to make improvements across different industry applications.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates an example network environment in accordance with one or more implementations.

FIG. 2 illustrates an example computing architecture for a system providing physiological predictions using machine learning in accordance with one or more implementations.

FIG. 3 is a schematic diagram illustrating an example machine learning model in accordance with one or more implementations.

FIG. 4 is a schematic diagram illustrating an example process for training one or more neural networks in accordance with one or more implementations.

FIG. 5 is a schematic diagram illustrating an example process for training an autoencoder in accordance with one or more implementations.

FIGS. 6A, 6B, and 6C illustrate various aspects of outputs of neural networks in accordance with one or more implementations.

FIG. 7 is a flow chart of an example process that may be performed for generating physiological predictions in accordance with one or more implementations.

FIG. 8 illustrates an example use case in which physiological predictions for a user are generated responsive to the user selecting a future workout in accordance with one or more implementations.

FIG. 9 illustrates an example use case in which physiological predictions for a user are used to recommend a workout for a user in accordance with one or more implementations.

FIG. 10 illustrates an electronic system with which one or more implementations of the subject technology may be implemented.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Machine learning has seen a significant rise in popularity in recent years due to the availability of training data, and advances in more powerful and efficient computing hardware. Machine learning may utilize models that are executed to provide predictions in particular applications.

The subject technology provides techniques for providing, using machine learning, physiological predictions, such as predictions of heart rate sequences (e.g., heartrates and/or heartrate ranges over time) and/or other physiological information for a user of an electronic device, from data captured by the electronic device or another electronic device of the user and/or other users (e.g., sensor data such as heartrate sensor data, inertial measurement unit data, magnetometer data, PPG data, optical sensor data, or the like, and/or wearable workout data such as steps, speed, elevation change, and/or weather information).

In some implementations, a differential equation model describing exercise physiology is integrated into a more flexible machine learning model that can be efficiently applied to various (e.g., tens, hundreds, millions) workouts and/or other activities, including workouts and/or other activities that have not previously been performed by the user. The resulting workout and subject representations may be used to predict heartrates, heartrate ranges, calories burned, and/or other physiological information for a user in previously unseen workouts and/or activities. As discussed in further detail hereinafter, the subject technology provides predictions of physiological data that are consistent with non-predictive measures of cardiorespiratory fitness.

In one or more implementations, a hybrid machine learning model is provided that combines a physiological model of heartrate and/or demand during exercise with neural network embeddings in order to learn user-specific fitness parameters. This model can be applied at scale to a large set of workout data collected, with user permission, from user devices (e.g., wearable devices). In one or more implementations, the disclosed hybrid machine learning model can accurately predict a heartrate response to exercise demand in new (e.g., previously unseen) workouts.

In one or more implementations, prior activity and/or other information gathered from a given user (e.g., using sensors of an electronic device of the given user and/or other devices and/or sensors) up to a time, t, can be used for training of a differential equation or hybrid model to predict one or more physiological signals for the given user at a time after the time, t (e.g., a future time that has not yet occurred).

In one or more implementations, prior activity and/or other information gathered from other users can be used for the training of the differential equations or hybrid model to predict physiological signals for a given user, even without using training data from the given user. In one or more implementations, training data for the training of the differential equations or hybrid model to predict physiological signals for a given user may include prior activity data for one or more other users, and non-activity information for the given user (e.g., for selecting demographically similar other users from which to obtain prior activity data for the training). Non-activity information may include age, sex, body mass index (e.g., BMI), and/or other demographic and/or biometric non-activity information. In this way, trained models as described herein can be provided that generate physiological predictions for users, whether or not the users have access to a device having activity sensors.

Implementations of the subject technology improve the ability of a given electronic device to provide sensor-based, machine-learning generated feedback to a user (e.g., a user of the given electronic device). These benefits therefore are understood as improving the computing functionality of a given electronic device, such as an end user device which may generally have less computational and/or power resources available than, e.g., one or more cloud-based servers.

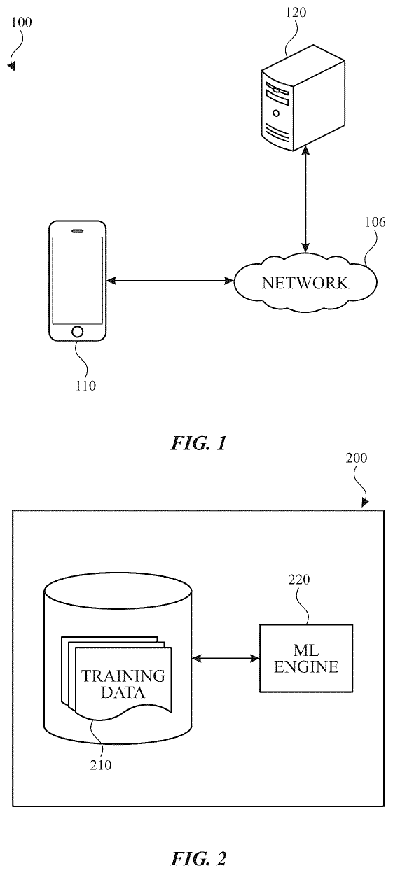

FIG. 1 illustrates an example network environment 100 in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

The network environment 100 includes an electronic device 110, and a server 120. The network 106 may communicatively (directly or indirectly) couple the electronic device 110 and/or the server 120. In one or more implementations, the network 106 may be an interconnected network of devices that may include, or may be communicatively coupled to, the Internet. For explanatory purposes, the network environment 100 is illustrated in FIG. 1 as including the electronic device 110, and the server 120; however, the network environment 100 may include any number of electronic devices and any number of servers.

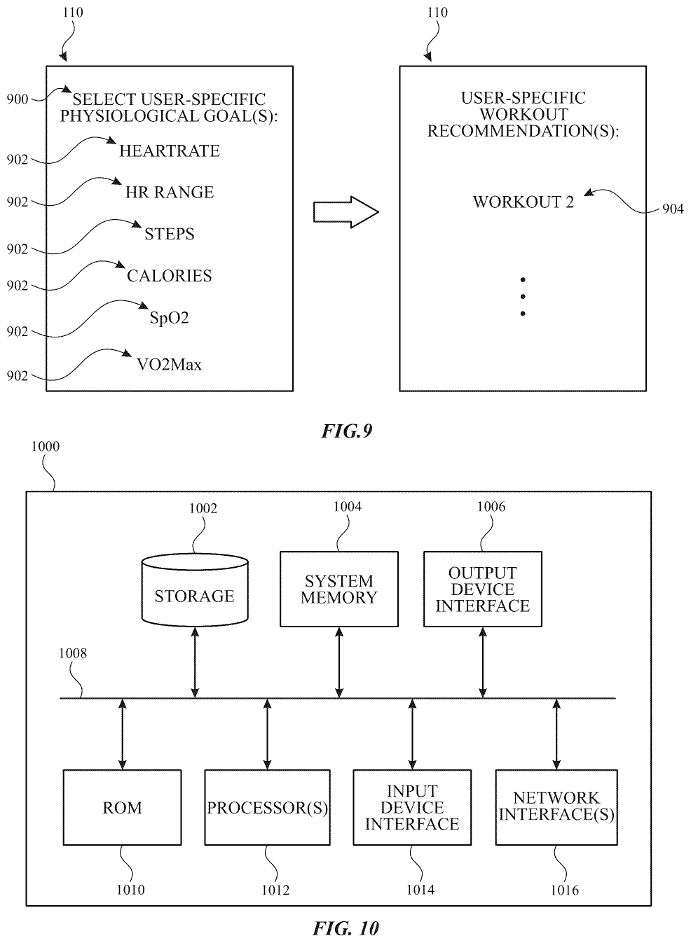

The electronic device 110 may be, for example, a desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 110 is depicted as a mobile electronic device (e.g., smartphone). The electronic device 110 may be, and/or may include all or part of, the electronic system discussed below with respect to FIG. 10.

In one or more implementations, the electronic device 110 may provide a system for training a machine learning model using training data, where the trained machine learning model is subsequently deployed to the electronic device 110. Further, the electronic device 110 may provide one or more machine learning frameworks for training machine learning models and/or developing applications using such machine learning models. In an example, such machine learning frameworks can provide various machine learning algorithms and models for different problem domains in machine learning. In an example, the electronic device 110 may include a deployed machine learning model that provides an output of data corresponding to a prediction or some other type of machine learning output. In one or more implementations, training and inference operations that involve individually identifiable information of a user of the electronic device 110 may be performed entirely on the electronic device 110, to prevent exposure of individually identifiable data to devices and/or systems that are not authorized by the user.

The server 120 may provide a system for training a machine learning model using training data, where the trained machine learning model is subsequently deployed to the server 120 and/or to the electronic device 110. In an implementation, the server 120 may train a given machine learning model for deployment to a client electronic device (e.g., the electronic device 110). In one or more implementations, the server 120 may train portions of the machine learning model that are trained using (e.g., anonymized) training data from a population of users, and the electronic device 110 may train portions of the machine learning model that are trained using individual training data from the user of the electronic device 110. The machine learning model deployed on the server 120 and/or the electronic device 110 can then perform one or more machine learning algorithms. In an implementation, the server 120 provides a cloud service that utilizes the trained machine learning model and/or continually learns over time.

In the example of FIG. 1, the electronic device 110 is depicted as a smartphone. However, it is appreciated that the electronic device 110 may be implemented as another type of device, such as a wearable device (e.g., a smart watch or other wearable device). The electronic device 110 may be a device of a user (e.g., the electronic device 110 may be associated with and/or logged into a user account for the user at a server). Although a single electronic device 110 is shown in FIG. 1, it is appreciated that the network environment 100 may include more than one electronic device, including more than one electronic device of a user and/or one or more other electronic devices of one or more other users.

FIG. 2 illustrates an example computing architecture for a system providing machine learning models, in accordance with one or more implementations. For explanatory purposes, the computing architecture is described as being provided by an electronic device 200, such as by a processor and/or memory of the server 120, or by a processor and/or a memory of any other electronic device, such as the electronic device 110. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

As illustrated, the electronic device 200 includes training data 210 for training a machine learning model. In an example, the server 120 may utilize one or more machine learning algorithms that uses training data 210 for training a machine learning (ML) model 220.

Training data 210 may include activity information associated with activities (also referred to as events), such as workouts. For example, the activity information may include workout measurements associated with workouts and/or other activities. The workout measurements may have been obtained over the course of multiple (e.g., many) prior workouts by a user of the electronic device 110, and/or by a population of other users, such as users that were wearing wearable devices during prior workouts and/or other activities, and authorized collection of anonymized workout measurements from the wearable devices. As an example, the training data 210 may include data from, e.g., hundreds or thousands of users and/or hundreds, thousands, or millions of workouts over the course of days, weeks, months or years. In one or more implementations, training data 210 may include training data obtained by a device on which the trained ML model 220 is deployed and/or training data obtained by other devices. In one or more implementations, workout measurements included in the training data 210 may include a number of steps, a horizontal speed (measured by a pedometer and/or a location sensor, such as a GPS sensor), an elevation change, a workout length in time or in distance, a heartrate, a blood oxygen level, and/or the like. Training data 210 may also include demographic information (e.g., age, gender, BMI, etc.) for a user of the electronic device 110, and/or a population of other users. Workout measurements may also include locations (e.g., an indoor location, an outdoor location, a geographical location such as a Global Positioning System (GPS) location, or other location information) of one or more portions of a workout or other activity and/or weather conditions at the time of a workout or other activity.

For example, in one or more implementations, the training data 210 may include workout measurements contributed anonymously from more than two hundred thousand workouts (e.g., outdoor runs) from more than seven thousand subjects over a period of three years. The workout data may include a heartrate during each workout as well as, for example four measures of the exercise intensity: a speed from a step sensor (e.g., a pedometer), a speed from a global positioning system (GPS) sensor, a step cadence, and an elevation gain. The sensor measurements may be interpolated on a grid (e.g., a ten second grid) to form, for each workout w, a heart rate time-series (and a multivariate time-series of exercise intensity where d is the duration of the workout. The workouts from which the workout data is obtained may be between fifteen and one hundred twenty minutes long, and the training data 210 may also contain weather information W at the time of each workout.

Machine learning model 220 may include one or more neural networks (e.g., including a latent variable model) combined with a solver for a physiological state equation, such as a heart rate dynamics equation, as described in further detail hereinafter.

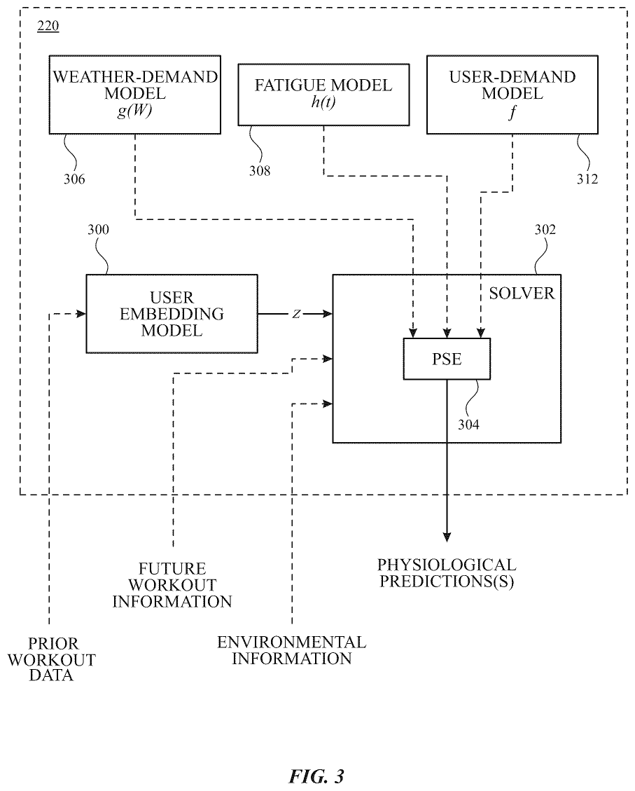

For example, FIG. 3 illustrates an implementation of the machine learning model 220 including one or more neural networks, and a solver for a physiological state equation that incorporates and/or is fed by the one or more neural networks. As illustrated in FIG. 3, the machine learning model 220 may include a user-embedding model 300 and a solver 302 for a physiological state equation (PSE) 304. As shown, the user-embedding model 300 may be trained to generate, based on prior workout data, an embedding, z, that is provided to the solver 302. The solver 302 may insert the embedding, z, into the PSE 304, and solve the PSE 304 to generate and output one or more physiological predictions. The prior workout data from which the embedding, z, is generated may be prior workout data for a user of the electronic device 110. However, in one or more use cases, prior workout data for the user of the electronic device 110 may not be available (e.g., in a case in which the electronic device 110 does not include sensors for obtaining workout data for a user, or in which no prior workout data has been previously obtained for the user of the electronic device 110). In one or more implementations, the prior workout data from which the embedding, z, is generated may be prior workout data for one or more other users of one or more other electronic devices. In one or more implementations, prior workout data for one or more other users of one or more other electronic devices may be supplemented and/or selected, for generation of the embedding, z, with demographic information (e.g., age, gender, BMI, etc.) for the user of the electronic device 110.

The user-embedding model 300 may be an encoder, e, and may be implemented as a neural network (e.g., a convolutional neural network, CNN). For example, the user-embedding model 300 may be a CNN with adaptive average pooling to accept variable input lengths. In one or more implementations, the embedding, z, may be a learned latent representation for a user (e.g., the user of the electronic device implementing the machine learning model 220, such as the electronic device 110).

In one or more implementations, the PSE 304 may be implemented as an ordinary differential equation, and may include one or more learned functions (e.g., functions having parameters that are learned by training a neural network). For example, the machine learning model 220 may include a user-demand model 312, a fatigue model 308, and a weather-demand model 306. For example, the user-demand model 312 maybe a function that translates an instantaneous activity intensity I into the necessary oxygen demand for that intensity I. For example, the fatigue model 308 may be a function that describes fatigue incurred over time, t, during a workout. For example, the weather-demand model 306 maybe a function that describes a change in oxygen demand as a function of one or more weather parameters, W, such as temperature or humidity.

In one or more implementations, the user-demand model 312, the fatigue model 308, and the weather-demand model 306 may each be implemented as a neural network. In this way, the parameters of functions ƒ, g, and h, respectively corresponding to the user-demand model 312, the fatigue model 308, and the weather-demand model 306, can be learned by training the respective neural networks using training data, such as training data 210 of FIG. 2. For example, the fatigue model 308 and the weather-demand model 306 may be trained using user population training data from a population of users other than the user of the electronic device 110. In one or more implementations, the user-demand model 312 may also be trained on the user population data from the population of users other than the user of the electronic device.

As shown, the solver 302 may generate one or more physiological prediction(s) for a particular user and for a particular future activity (e.g., a particular future workout) responsive to receiving, as inputs, future workout information. For example, the future workout information may be parameters of a workout (e.g., a run, a swim, a gym activity, etc.) that has not yet been performed by the user of a device implementing the machine learning model 220, and may include route information, elevation information, water current information, and/or any other information that describes characteristics of the future workout. In one or more implementations, future workout information may include future workout information for multiple different future workouts and/or multiple variations of a future workout (e.g., variations of a running, cycling, hiking, walking, or swimming route), so that physiological predictions for the multiple different future workouts and/or multiple variations of a future workout can be used to recommend a custom workout (or custom list of workouts the user can select from) for which the user is predicted to achieve a desired (e.g., user input) heartrate, workout time, or other activity level. The future workout information may be information obtained from other users that have previously performed the workout that has not yet been performed by the user of the device implementing the machine learning model 220, and/or from map data or other stored data (e.g., user-agnostic data) describing the future workout. As shown in FIG. 3, the solver 302 may also be provided with environmental information, such as weather information for the future workout. For example, the environmental information may include a current temperature at the location of the future workout, a current humidity at the location of the future workout, a predicted temperature at the location of the future workout, a predicted humidity at the location of the future workout, a median or average elevation at the location of the future workout, and/or other environmental and/or weather information.

In one or more other implementations, the solver 302 may generate a deterministic solution to the PSE 304. For example, in one or more implementations, the solver 302 may solve the PSE 304 using an iterative operation (e.g., a Fourth Order Runge-Kutta method) to generate the physiological prediction(s) responsive to receiving the embedding, z, the future workout information, and/or the environmental information (e.g., such that the PSE 304 yields an solution that is differentiable against its input parameters). In other implementations, the solver 302 may be implemented as a neural network trained to generate the physiological prediction(s) responsive to receiving the embedding, z, the future workout information, and/or the environmental information.

In one or more use cases, a user may select a future activity, such as a future workout to be performed. Responsive to the selection of the future workout, activity information (e.g., future workout information) and/or environmental information for that future workout may be provided to the solver 302. Responsive to the selection of the future workout, the embedding, z, may also be provided to the solver 302. The embedding, z, may be determined at the time of the selection of the future workout, may be generated, based on prior activity information for the user of the electronic device 110, prior to the selection of the future workout and stored (e.g., at the electronic device 110), and/or may be generated based on prior activity information for the future workout from one or more other users. The solver 302 may then insert the embedding, z, the future workout information, and/or the environmental information into the PSE 304, and solve the PSE 304 to generate the physiological prediction(s). For example, the embedding, z, may be inserted into the user-demand model 312, and the solver 302 may solve the PSE 304, wherein the PSE 304 includes: the user-demand model 312 with the embedding, z, for the user; the fatigue model 308; and/or the weather-demand model 306, for the future workout corresponding to the future workout information.

In one or more implementations, heartrate dynamics in response to exercise can be described by ordinary differential equations (ODEs). These ODE approaches translate the physical mechanisms of the human body into differential equations in order to incorporate domain knowledge in the modeling. One approach introduces a body oxygen demand D as an intermediary quantity to link an exercise intensity I and the heartrate HR through a coupled ODE, such as the system of Equations 1 below:

$$\{ \begin{matrix}\dot{D} ( t ) = B \cdot ( f ( I ( t ) ) - D ( t ) ) \\ \dot{HR} ( t ) = A \cdot {( HR ( t ) - {HR}_{min} )}^{α} \cdot {( {HR}_{max} - HR ( t ) )}^{β} \cdot ( D ( t ) - HR ( t ) ) \\ HR ( 0 ) = {HR}_{0} and D ( 0 ) = {D}_{0}\end{matrix} . \qquad ( 1 )$$

In this dynamical system, ƒ may be a function translating the instantaneous activity intensity I into the necessary oxygen demand for I. In the system of Equations 1 above, the top equation attempts to match the current body oxygen demand D with the instantaneous demand ƒ(I). Parameter B controls how fast D adapts to ƒ(I). At the same time, the second equation drives the heart rate, HR, toward the pace required to deliver the demand D. Parameter A controls how fast the heart can adapt, and the elements HRmin, HRmax, α, and β control how difficult it is to reach the maximal heart rate or to rest down to the minimal heart rate.

However, the ODE shown in Eqns. 1 above can be difficult to apply to large scale uncontrolled environments and to model workout data from user devices. Moreover, in the explicit form of the functions in the Eqns. 1 above, the function, ƒ, may be limited to simple functions.

Aspects of the subject technology provide a hierarchical model (e.g., a hybrid machine learning model, such as the machine learning model 220) that relates the ODE parameters together. This hierarchical model can facilitate a large scale applicability of the technology based on identifications of correlations between the ODE parameters across individuals, including their evolution over time. Because learned parameters capture the heartrate response to exercise, they can be interpreted as summarizing the fitness level and cardio-respiratory health of various users.

In one or more implementations, in order to generate the machine learning model 220, the health state of individual, i, at date, T, can be represented by a low dimensional latent vector Z_{i,T}∈ [symbol] . One or more (e.g., each) of the ODE parameters can then be a function of this representation, z. Each parameter's function, as well as the function ƒ may then be learned as neural networks. In one or more implementations, the physical model represented by the ODE may also be modified to incorporate (i) the effect of weather, W (e.g., temperature, humidity, and/or other weather and/or environmental parameters) into the demand equation, ƒ, and/or (ii) the fatigue, h, incurred over time t during a future workout. For instance, higher temperatures can induce a higher oxygen demand in some use cases. The weather and/or fatigue effects can also be parameterized by neural networks g(W) and h(t). For a health state, z, of a user, and an intensity t→I(t), the heartrate response (in weather W) may be governed by the system of Equations 2 below:

$$\{ \begin{matrix}\dot{D} ( t ) = B ( z ) \cdot ( f ( I ( t ) , z ) \cdot g ( W ) \cdot h ( t ) - D ( t ) ) \\ \begin{matrix}\dot{HR} ( t ) = ( z ) \cdot {( HR ( t ) - {HR}_{min} ( z ) )}^{α ( z )} \cdot \\ {( {HR}_{max} ( z ) - HR ( t ) )}^{β ( z )} \cdot ( D ( t ) - HR ( t ) )\end{matrix} \\ HR ( 0 ) = {HR}_{0} ( z ) and D ( 0 ) = {D}_{0} ( z )\end{matrix} . \qquad ( 2 )$$

For example, the system of Equations 2 above may form the PSE 304 of FIG. 3, in which g(W) corresponds to the weather-demand model 306, h(t) corresponds to the fatigue model 308, and ƒ(I(t),z) corresponds to the user-demand model 312 and is a function of z. In order to generate physiological predictions, the solver 302 may solve the system of Equations 2 above.

In order, for example, to infer a user's health representations outside the training set, and be able to incorporate the evolution of the user's health over time to use the PSE 304 (e.g., Equations 2) to predict future heart rates, the user-embedding model 300 may be used to generate z as an embedding. For example, the user-embedding model 300 may be implemented as an amortized auto-encoder schema that concatenates user i's workout history up to T and encodes that history into a health representation z_{i,T}:

z_{i,T}=e(HR^{(o)}, I^{(o)} . . . , I_{(ω*)}HR^{(ω*)}), (3)

where w* is the last workout before date T. In this example, the embedding, z, is generated based on prior user activity data (e.g., prior workout data) for the user, i, for which the prediction is being made. However, in other examples, the embedding, z, may also, or alternatively, be generated based on prior activity data (e.g., prior workout data) from one or more other users (e.g., along with demographic information for the user).

As discussed herein, the weather-demand model 306 (e.g., function g(W)), the fatigue model 308 (e.g., function h(t)), and/or the user-demand model 312 (e.g., function ƒ) can be implemented as neural networks to learn the parameters of the respective functions.

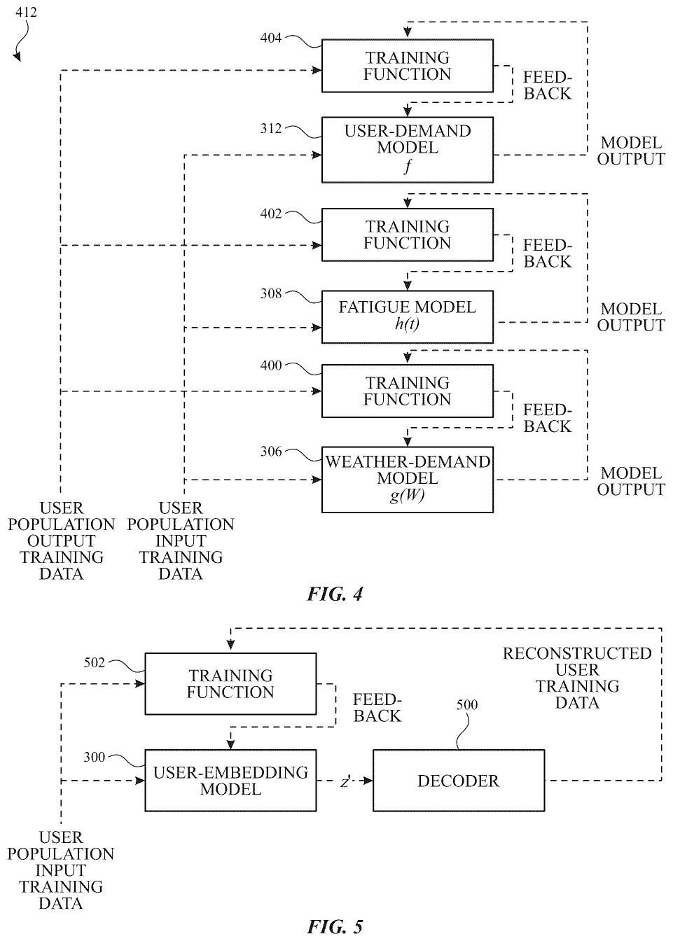

FIG. 4 illustrates an example training operation for the weather-demand model 306 (e.g., function g(W)), the fatigue model 308 (e.g., function h(t)), and/or the user-demand model 312 (e.g., function ƒ). In the example of FIG. 4, user population input training data (e.g., training data 210 from a population of users which may include or exclude the user of the device implementing the trained machine learning model 220) may be provided as training inputs to the weather-demand model 306 (e.g., a neural network representing the function g(W)), the fatigue model 308 (e.g., a neural network representing the function h(t)), and/or the user-demand model 312 (e.g., a neural network representing the function ƒ). As shown, user population output training data (e.g., labels) from the user population training data may be provided to a training function for each of the weather-demand model 306, the fatigue model 308, and/or the user-demand model 312.

In the example of FIG. 4, the user-demand model 312 generates, responsive to receiving the input training data from the user population training data, a model output that is provided to a training function 404 (e.g., a cost function). In this example, the training function 404 may compare the model output from the user-demand model 312 to corresponding output training data from the user population training data, and may provide feedback, based on the comparison, to the user-demand model 312. One or more weights and/or other parameters of the user-demand model 312 may then be adjusted based on the feedback from the training function 404. In the example of FIG. 4, the fatigue model 308 generates, responsive to receiving the input training data from the user population training data, a model output that is provided to a training function 402 (e.g., a cost function). In this example, the training function 402 may compare the model output from the fatigue model 308 to corresponding output training data from the user population training data, and may provide feedback, based on the comparison, to the fatigue model 308. One or more weights and/or other parameters of the fatigue model 308 may then be adjusted based on the feedback from the training function 402. In the example of FIG. 4, the weather-demand model 306 generates, responsive to receiving the input training data from the user population training data, a model output that is provided to a training function 400 (e.g., a cost function). In this example, the training function 400 may compare the model output from the weather-demand model 306 to corresponding output training data from the user population training data, and may provide feedback, based on the comparison, to the weather-demand model 306. One or more weights and/or other parameters of the weather-demand model 306 may then be adjusted based on the feedback from the training function 400.

FIG. 5 illustrates an example training operation for the user-embedding model 300 (e.g., e). In the example of FIG. 5, user population training data (e.g., training data 210 from a population of users which may include or exclude the user of the device implementing the trained machine learning model 220) may be provided as training input to the user-embedding model 300. In the example of FIG. 5, the user-embedding model 300 generates, responsive to receiving the input training data from the user training data, an embedding, z′, that is provided to a decoder 500. The decoder 500 may generate a reconstruction of the user population training data that was encoded into the embedding, z′. The reconstructed user training data may be provided to a training function 502 (e.g., a cost function). In this example, the training function 502 may compare the reconstructed user training data from the decoder 500 to the user input training data from which the embedding, z′, was generated, and may provide feedback, based on the comparison, to the user-embedding model 300. One or more weights and/or other parameters of the user-embedding model 300 may then be adjusted based on the feedback from the training function 502.

In the examples of FIGS. 4 and 5, each of the models is trained using a separate cost function for that model. However, this is merely illustrative, and, in one or more implementations, a joint cost function may be used (e.g., to simultaneously train two, three, or all of the user-embedding model 300, the weather-demand model 306, the fatigue model 308, and/or the user-demand model 312), and/or other joint training operations may be performed. For example, for training the machine learning model 220, the representation encoding with CNN followed by the heart rate ODE decoding may be chained and a Gaussian likelihood may be computed to form an objective function on which a gradient descent can be run. In one or more implementations, batches or portions of workouts may be sub-sampled, and stochastic gradient descent can be performed. In this way, the gradient updates may simultaneously learn the representation encoder, and all of the ODE internal neural networks parameters.

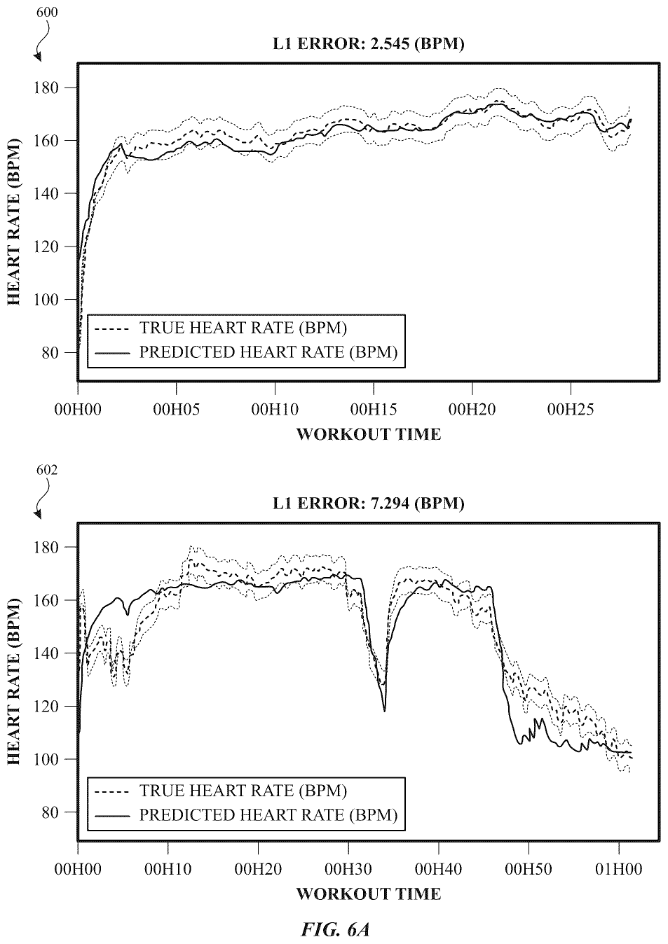

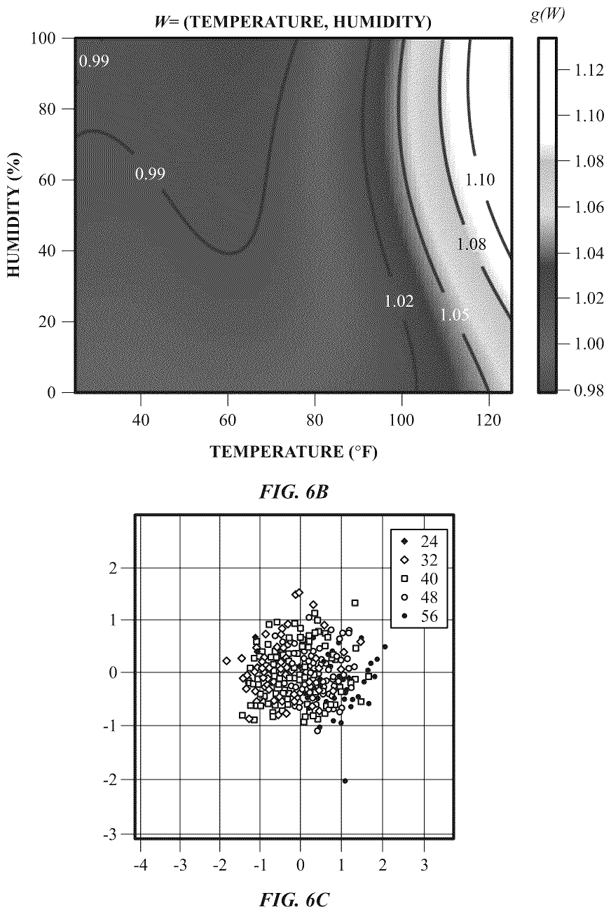

Using the trained machine learning model 220, the representation, Z_{i,T}, encoding an individual's workout history (or a workout history of demographically similar users having a given or learned measure of similarity with the individual) can be used to predict a heartrate, a heartrate zone, and/or other physiological information for the user in future workouts. The accuracy of heartrate prediction can be determined using workouts that were held-out for each subject. FIG. 6A shows two examples 600 and 602 comparing a true heart rate to the heart rate predicted using the machine learning model 220. Note that, for predicting a heart rate for a workout w happening at date T, the machine learning model 220 may only use the workout intensity measures of that sample I(w) and the health representation Z_{i,T} from encoding the previous workouts (e.g., no heartrate measurements HR(w) are observed by the model for making the predictions).

A metric for estimating the accuracy of the disclosed model is the predictive performance of the model in estimating the heartrate, HR, after an initial warmup period of the workout. Indeed, the disclosed model predicts a starting heart rate, HR0, and demand, D0, from the representation, z, but these quantities depend on the user activity preceding the workout, which is typically not known at inference/prediction time. The disclosed model has been shown to adapt to varying preceding user activity levels.

In one or more implementations, physiological predictions from the machine learning model 220 can include predictions of a number of calories that will be burned during a workout. For example, a calories-burned prediction can be derived from predicted heartrates during the workout with a linear formula. Providing predicted numbers of calories burned can be useful for planning workouts based on calories burned goals, and even more useful in cases where individuals performing a workout are not wearing a wearable device that records a heartrate. It has been shown that the machine learning model 220 can reliably estimate the amount of calories burned with, for example, a 5% relative error (e.g., which may be the same or similar relative error as the heartrate predictions), including in use cases in which only workout metrics that can be measured using a smartphone are used.

In one or more implementations, physiological predictions from the machine learning model 220 can include predictions of heartrate zones and/or heartrate ranges (e.g., a predicted maximum heartrate and a predicted minimum heartrate during a workout). For example, a heartrate zone may be the percentage of an individual's maximum heartrate reached throughout the course of an exercise. Predictions of heartrate zones and/or heartrate ranges can help individuals plan personalized exercise routines to more effectively achieve their fitness goals. Defining six zones (e.g., percent intervals [0, 50, 60, 70, 80, 90, 100]) of maximum heartrate, Table 2 shows the performance of the disclosed models on predicting the heartrate zone for the whole population, as well as different subgroups of the population. In one or more implementations, a heartrate range may be generated from a time series of the predicted heartrates. In one or more other implementations, the heartrate range may be predicted directly from z (e.g., without predicting individual heartrates at individual times).

TABLE 2
Predictive performance of heart rate zones

| | Population cohort | | |
|---|---|---|---|
| | Full cohort | Female individuals | Male individuals |
| Accuracy | 68.11 ± 21.73 | 67.96 ± 21.91 | 68.51 ± 21.07 |

Leveraging the interpretability of the ODE model defined by Eqns. 2 above, the impact of the weather on heart rate can be quantified by analyzing the learned neural network, g, and quantifying the relative effect of weather on the body oxygen demand. As shown in FIG. 6B, an increase in body oxygen demand by up to ten percent can occur and can be predicted for high temperatures and/or humidity.

A metric of cardiorespiratory fitness called the VO2Max measure can be used to show that the learned representations disclosed herein summarize information about cardiorespiratory health. VO2Max is the maximum amount of oxygen the body can consume during exercise. This value can be measured using the heart and motion sensors on wearable devices and using demographic information such as age, biological sex, weight, and height. Using the health representations Z_{i,T}, the VO2Max can be predicted, for comparison with measured VO2Max, using a linear regression model with an accuracy of, for example, ±3 mL/(kg·min). Table 3 reports these results and compares the performance of the predictions from the learned representations with those obtained using demographics alone, and combined. FIG. 6C shows a 2D projection of the health representation, z, for different workouts where the separation of higher and lower values of VO2Max (indicated by the dot style) can be seen.

TABLE 3
VO2Max prediction performance

| | MSE | MAE | R2-score | Explained variance |
|---|---|---|---|---|
| Demographics only | 30.53 ± 0.09 | 4.36 ± 0.006 | 0.36 ± 0.002 | 0.36 ± 0.002 |
| ODE representations | 16.2 ± 0.11 | 3.07 ± 0.01 | 0.66 ± 0.002 | 0.66 ± 0.002 |
| Both | 8.9 ± 0.10 | 2.16 ± 0.007 | 0.81 ± 0.003 | 0.81 ± 0.003 |

In one or more implementations, the physiological predictions generated by the machine learning model 220 may include a prediction or a warning of a potential cardiovascular event (e.g., a heart attack or low blood oxygen level) that could occur during a workout (e.g., by comparing a predicted heartrate to a heartrate-risk threshold). In one or more implementations, the physiological predictions generated by the machine learning model 220 may be used to track a user's fitness level over time, provide personalized workout planning, and/or predict changes in cardiovascular health (e.g., including detecting a potential health or fitness deterioration or other issue).

In the examples described herein, the learned latent representation, z, is generated by a neural network by providing historical data of the user to user embedding model (e.g., the user-embedding model 300, such as an autoencoder), and obtaining the learned latent representation, z, as an output of the user embedding model. In one or more other implementations, rather than generating the representations, z, using a neural network (e.g., the user-embedding model 300) as described above, the representations, z, may be generated by fitting a set of free parameters of the representation, z, to each workout, and using a Gaussian process to correlate the fitted parameters across workouts to each other for a single subject (user).

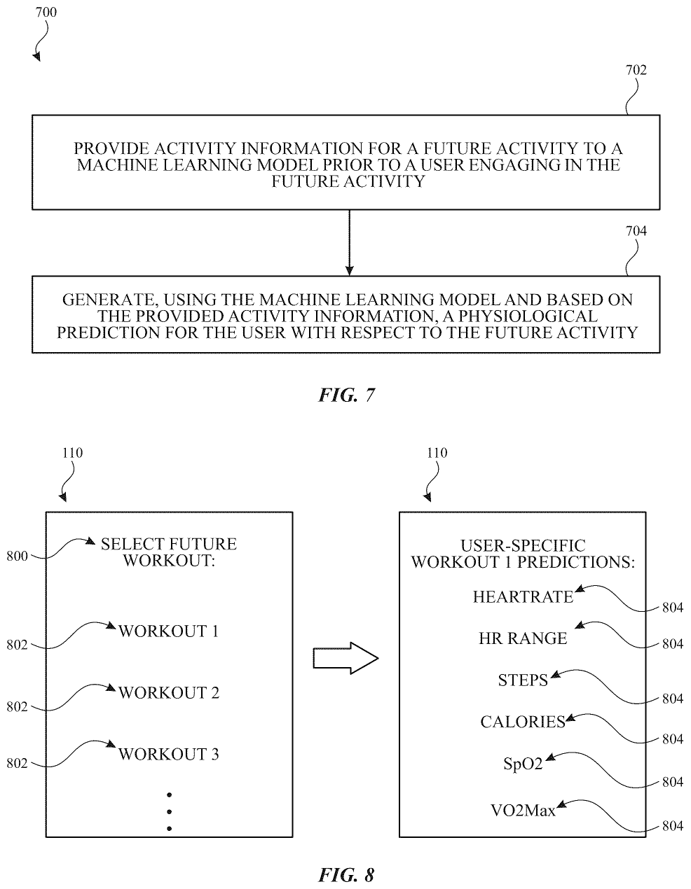

FIG. 7 illustrates a flow diagram of an example process 700 that may be performed for generating physiological predictions, in accordance with implementations of the subject technology. For explanatory purposes, the process 700 is primarily described herein with reference to the electronic device 110 of FIG. 1. However, the process 700 is not limited to the electronic device 110 of FIG. 1, and one or more blocks (or operations) of the process 700 may be performed by one or more other components of other suitable devices and/or servers. Further for explanatory purposes, some of the blocks of the process 700 are described herein as occurring in serial, or linearly. However, multiple blocks of the process 700 may occur in parallel. In addition, the blocks of the process 700 need not be performed in the order shown and/or one or more blocks of the process 700 need not be performed and/or can be replaced by other operations.

As illustrated in FIG. 7, at block 702, activity information (e.g., future workout information) for a future activity (e.g., a future workout) may be provided to a machine learning model (e.g., machine learning model 220) prior to a user engaging in the future activity, where the machine learning model has been trained to output physiological predictions for the user based at least in part on prior physiological data (e.g., included in the training data 210) obtained during the user engaging in prior activities different from the future activity. For example, activity information (e.g., future workout information) for a future activity (e.g., a future workout) may be provided to a machine learning model (e.g., machine learning model 220) that has been trained to output physiological predictions for a user engaging in activities. For example, the activity information may include activity information obtained from other users that have previously performed the future activity that has not been performed by the user, or may be obtained from map data or other data describing the activity. For example, in a use case in which the future activity is a run, a hike, or a walk, the activity information may include, or may be derived from, a map of the run, hike, or walk. For example, the activity information may include an elevation at each point on a route corresponding to the run, hike, or walk.

In one or more implementations, the machine learning model may include a user embedding model (e.g., user-embedding model 300) that generates a learned latent representation (e.g., z) for the user, learned based at least on training data including historical activity information and physiological information for the user. For example, the historical activity information may include information describing prior activities (e.g., workouts), different from the future activity, performed by the user. The historical physiological information for the user may include heartrates, blood oxygen levels, steps, speeds, calories, or other physiological information obtained by an electronic device (e.g., electronic device 110 or another electronic device such as a heartrate monitor) while the user performed the prior activities. In one or more other implementations, the historical physiological information for the user may include non-activity information, such as an age, a gender, a BMI, or the like, which may be input by the user or sensed by the electronic device. In one or more implementations, the user embedding model (e.g., user-embedding model 300) may generate a learned latent representation (e.g., z) for a user that is similar to the user, learned based at least on training data including historical activity information and physiological information for other similar users (e.g., other users determined to be demographically similar based on the user's non-activity information).

At block 704, a physiological prediction for the user may be generated with respect to the future activity using the machine learning model and based on the provided activity information. In one or more implementations, the physiological prediction may be generated by the machine learning model at an electronic device (e.g., electronic device 110) of the user. In one or more implementations, the machine learning model may also include a solver (e.g., solver 302). Generating the physiological prediction may include providing the activity information and the learned latent representation for the user to the solver, and generating the physiological prediction with the solver by solving a physiological state equation using the learned latent representation for the user and the activity information (e.g., as described herein in connection with FIG. 3).

In one or more use cases, the future activity includes a workout, the activity information includes workout parameters (e.g., future workout information as described herein). In one or more use cases, the physiological prediction may include a predicted heart rate zone for the user during the workout, a predicted heart rate for the user during the workout, a predicted number of calories that will be burned by the user during the workout, and/or a prediction of a potential cardiovascular event for the user during the workout.

In one or more implementations, the machine learning model may include a user-demand model (e.g., ƒ, which may be user-demand model 312), a fatigue model (e.g., h(t), which may be fatigue model 308), and a weather-demand model (e.g., g(W), which may be weather-demand model 306). In one or more implementations, the process 700 may also include training the machine learning model using workout measurements for a population of users (e.g., user population input training data and/or user population output training data). Training the machine learning model may include training the user-demand model, the fatigue model, and the weather-demand model (e.g., as described herein in connection with FIG. 4). Training the machine learning model may include adjusting one or more weights and/or other parameters of one or more neural networks of the machine learning model (e.g., as described herein in connection with FIGS. 4 and/or 5).

For example, in one or more implementations, the machine learning model may include a trained user-demand model, a trained fatigue model, and a trained weather-demand model. The solver may be configured to solve a physiological state equation, in part, by inserting the trained user-demand model, the trained fatigue model, and the trained weather-demand model (e.g., and a learned embedding, z) into the physiological state equation.

In one or more implementations, the process 700 may also include providing environmental information for the future activity to the machine learning model, and the physiological prediction may be based in part on the environmental information. As examples, the environmental information may include a location of the future activity, a temperature, a humidity, other weather information, or weather quality information. For example, the environmental information may include a current or predicted temperature at the location, a current or predicted humidity at the location, and/or other weather information, or weather quality information.

In various implementations, physiological predictions can be made for workouts selected by a user, and/or physiological predictions can be used to suggest a workout for a user based on physiological goals provided by a user.

For example, FIG. 8 illustrates a use case in which the electronic device 110 provides an option 800 for a user to select one of a set of future workouts 802. For example, the future workouts 802 may be workouts that have not been previously performed by the user of the electronic device 110. The future workouts 802 may be workouts with parameters (e.g., future workout information) that are known or obtainable by the electronic device 110. For example, the future workouts 802 may correspond to runs, walks, hikes, swims, or cycles on a known route (e.g., a route having a route map that is stored at or accessible by the electronic device 110), gym activities, and/or any other workouts for which parameters (e.g., future workout information) are known or obtainable by the electronic device 110. As illustrated in FIG. 8, the user may select one of the future workouts 802 (e.g., WORKOUT 1). Responsively, the electronic device 110 may generate, using the machine learning model 220, one or more physiological predictions 804 for the selected workout for the user of the electronic device 110. As shown, the physiological predictions 804 may include a heartrate, a heartrate range, a number of steps, a number of calories burned, a blood-oxygen level (SpO2), and/or a VO2Max that may be experienced by the user of the electronic device 110 while performing the selected workout. In various implementations, the physiological predictions 804 may include single prediction for the overall selected workout, or may include predictions as a function of time or as a function of distance during the selected workout.

In one illustrative use case, the user of a device implementing the machine learning model 220 may be visiting a new city and considering going for a run in the new city. In one or more implementations, an embedding, z, for the user may have already been learned at the device of the user. Prior to going for the run, the user may select the run (e.g., by selecting an indication of a route corresponding to the run), and the device of the user may, responsively, obtain parameters of the run (e.g., a distance, an elevation change, a route map) and/or weather information (e.g., a humidity and/or a temperature). The device of the user may then provide the embedding, z, and the parameters of the run (and/or the weather information) to machine learning model 220. The solver 302 may then solves the PSE 304 into which the embedding, z, and the parameters of the run (and/or the weather information) have been inserted, to generate the physiological predictions 804.

FIG. 9 illustrates another example use case in which the electronic device 110 provides an option 900 for a user to select and/or input one or more physiological goals 902. For example, the user of the electronic device 110 may input a target heart rate, a target heartrate range, a target number of steps, a target number of calories, a target blood oxygen level, and/or a target VO2Max. Responsively, the electronic device 110 may, using the machine learning model 220, generate physiological predictions for one or more available workouts (e.g., workouts in the vicinity of the user). The electronic device 110 may then provide a recommendation 904 for one (or more) of the available workouts for which the physiological predictions match the user's physiological goals 902. For example, in one or more implementations, the recommendation 904 may be provided by a mapping application, and may be presented in the form of a personalized route that will bring the user's workout to the user's desired activity and/or physiological level(s). For example, a user may provide a request to the mapping application to provide a map of one or more (e.g., nearby) routes that will cause the user to exercise for a user-selected number of minutes at the user's normal running heartrate. In another example, the recommendation 904 may be provided by a fitness application (e.g., in a list of recommended workouts for a particular user-requested level of heartrate and/or effort, or in a list of recommended workouts that are categorized by the level of heartrate and/or effort the user is predicted to achieve during the workout).

Although physiological predictions for workouts are described herein in connection various examples, physiological predictions can be provided for activates other than workouts, such as climbing a flight of stairs, flying on an airplane, scuba diving, performing a dance, or any other physical activity.

The increased availability of wearable devices empowers individuals to track their health. The subject technology may help to quantify this measure through modelling the heart rate response to workouts. Learned representations that summarize the dynamics of the HR response can serve as a measure for an individual's cardiorespiratory fitness. This measure can help track fitness level over time, provide personalized workout planning, and predict changes in cardiovascular health.

As described above, one aspect of the present technology is the gathering and use of data available from specific and legitimate sources for generating physiological predictions. The present disclosure contemplates that in some instances, this gathered data may include personal information data that uniquely identifies or can be used to identify a specific person. Such personal information data can include audio data, demographic data, location-based data, online identifiers, telephone numbers, email addresses, home addresses, biometric data or records relating to a user's health or level of fitness (e.g., vital signs measurements, medication information, exercise information, motion information, heartrate information workout information), date of birth, or any other personal information.

The present disclosure recognizes that the use of such personal information data, in the present technology, can be used to the benefit of users. For example, the personal information data can be used for generating physiological predictions.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users, and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates aspects in which users selectively block the use of, or access to, personal information data. That is, the present disclosure contemplates that hardware and/or software elements can be provided to prevent or block access to such personal information data. For example, in the example of generating physiological predictions, the present technology can be configured to allow users to select to “opt in” or “opt out” of participation in the collection and/or sharing of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level or at a scale that is insufficient for facial recognition), controlling how data is stored (e.g., aggregating data across users), and/or other methods such as differential privacy.

Therefore, although the present disclosure broadly covers use of personal information data to implement one or more various disclosed implementations, the present disclosure also contemplates that the various implementations can also be implemented without the need for accessing such personal information data. That is, the various implementations of the present technology are not rendered inoperable due to the lack of all or a portion of such personal information data.

FIG. 10 illustrates an electronic system 1000 with which one or more implementations of the subject technology may be implemented. The electronic system 1000 can be, and/or can be a part of, the electronic device 100, and/or the server 120 shown in FIG. 1. The electronic system 1000 may include various types of computer readable media and interfaces for various other types of computer readable media. The electronic system 1000 includes a bus 1008, one or more processing unit(s) 1012, a system memory 1004 (and/or buffer), a ROM 1010, a permanent storage device 1002, an input device interface 1014, an output device interface 1006, and one or more network interfaces 1016, or subsets and variations thereof.

The bus 1008 collectively represents all system, peripheral, and chipset buses that communicatively connect the numerous internal devices of the electronic system 1000. In one or more implementations, the bus 1008 communicatively connects the one or more processing unit(s) 1012 with the ROM 1010, the system memory 1004, and the permanent storage device 1002. From these various memory units, the one or more processing unit(s) 1012 retrieves instructions to execute and data to process in order to execute the processes of the subject disclosure. The one or more processing unit(s) 1012 can be a single processor or a multi-core processor in different implementations.

The ROM 1010 stores static data and instructions that are needed by the one or more processing unit(s) 1012 and other modules of the electronic system 1000. The permanent storage device 1002, on the other hand, may be a read-and-write memory device. The permanent storage device 1002 may be a non-volatile memory unit that stores instructions and data even when the electronic system 1000 is off. In one or more implementations, a mass-storage device (such as a magnetic or optical disk and its corresponding disk drive) may be used as the permanent storage device 1002.

In one or more implementations, a removable storage device (such as a floppy disk, flash drive, and its corresponding disk drive) may be used as the permanent storage device 1002. Like the permanent storage device 1002, the system memory 1004 may be a read-and-write memory device. However, unlike the permanent storage device 1002, the system memory 1004 may be a volatile read-and-write memory, such as random access memory. The system memory 1004 may store any of the instructions and data that one or more processing unit(s) 1012 may need at runtime. In one or more implementations, the processes of the subject disclosure are stored in the system memory 1004, the permanent storage device 1002, and/or the ROM 1010. From these various memory units, the one or more processing unit(s) 1012 retrieves instructions to execute and data to process in order to execute the processes of one or more implementations.

The bus 1008 also connects to the input and output device interfaces 1014 and 1006. The input device interface 1014 enables a user to communicate information and select commands to the electronic system 1000. Input devices that may be used with the input device interface 1014 may include, for example, alphanumeric keyboards and pointing devices (also called “cursor control devices”). The output device interface 1006 may enable, for example, the display of images generated by electronic system 1000. Output devices that may be used with the output device interface 1006 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 10, the bus 1008 also couples the electronic system 1000 to one or more networks and/or to one or more network nodes, such as the electronic device 110 shown in FIG. 1, through the one or more network interface(s) 1016. In this manner, the electronic system 1000 can be a part of a network of computers (such as a LAN, a wide area network (“WAN”), or an Intranet, or a network of networks, such as the Internet. Any or all components of the electronic system 1000 can be used in conjunction with the subject disclosure.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include”, “have”, or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method comprising:
providing activity information for a future activity to a machine learning model prior to a user engaging in the future activity, wherein the machine learning model has been trained to output physiological predictions for the user based at least in part on prior physiological data associated with prior activities performed prior to the future activity; and
generating, using the machine learning model and based on the provided activity information, a physiological prediction for the user with respect to the future activity.

2. The method of claim 1, wherein the machine learning model comprises a user embedding model that generates a learned latent representation for the user, learned based at least on training data comprising historical activity information.

3. The method of claim 2, wherein the historical activity information comprises historical activity information for the user, and wherein the prior activities are each different from the future activity.

4. The method of claim 2, wherein the historical activity information comprises historical activity information for another user different from the user.

5. The method of claim 2, wherein the machine learning model further comprises a solver, and wherein generating the physiological prediction comprises:
providing the activity information and the learned latent representation for the user to the solver; and
generating the physiological prediction with the solver by solving a physiological state equation using the learned latent representation for the user and the activity information.

6. The method of claim 1, wherein the future activity comprises a workout, and wherein the activity information comprises workout parameters.

7. The method of claim 6, wherein the physiological prediction comprises a predicted heartrate zone for the user during the workout.

8. The method of claim 6, wherein the physiological prediction comprises a predicted heartrate for the user during the workout.

9. The method of claim 6, wherein the physiological prediction comprises a predicted number of calories that will be burned by the user during the workout.

10. The method of claim 6, wherein the physiological prediction comprises a prediction of a potential cardiovascular event for the user during the workout.

11. The method of claim 6, further comprising training the machine learning model to generate the physiological predictions using workout measurements for a population of users, wherein training the machine learning model comprises training a user-demand model, a fatigue model, and a weather-demand model.

12. The method of claim 1, further comprising:
providing environmental information for the future activity to the machine learning model, wherein the physiological prediction is based in part on the environmental information.

13. The method of claim 12, wherein the environmental information comprises a location of the future activity, a temperature, a humidity, or other weather information.

14. A device, comprising:
a memory; and
one or more processors configured to:
provide activity information for a future activity to a machine learning model prior to a user engaging in the future activity, wherein the machine learning model has been trained to output physiological predictions for the user based at least in part on prior physiological data associated with prior activities performed prior to the future activity; and
generate, using the machine learning model and based on the provided activity information, a physiological prediction for the user with respect to the future activity.

15. The device of claim 14, wherein the machine learning model comprises a user embedding model configured to generate a learned latent representation for the user, learned based at least on training data comprising physiological information for the user.

16. The device of claim 15, wherein the machine learning model further comprises a solver, and wherein the one or more processors are configured to generate the physiological prediction at least in part by:
providing activity information and the learned latent representation for the user to the solver; and
generating the physiological prediction with the solver by solving a physiological state equation using the learned latent representation for the user and the activity information.

17. The device of claim 16, wherein the machine learning model further comprises a trained user-demand model, a trained fatigue model, and a trained weather-demand model.

18. The device of claim 17, wherein the solver is configured to solve the physiological state equation, in part, by inserting the trained user-demand model, the trained fatigue model, and the trained weather-demand model into the physiological state equation.

19. A non-transitory machine-readable medium comprising code that, when executed by a processor, causes the processor to perform operations comprising:
providing activity information for a future activity to a machine learning model that has been trained to output physiological predictions for a user, prior to the user engaging in the future activity; and
generating, using the machine learning model and based on the provided activity information, a physiological prediction for the user with respect to the future activity.

20. The non-transitory machine-readable medium of claim 19, wherein the machine learning model comprises a user embedding model configured to generate a learned latent representation for the user, learned based at least on training data comprising historical activity information for the user.

21. The non-transitory machine-readable medium of claim 19, wherein generating the physiological prediction for the user comprises generating the physiological prediction for the user based at least in part on representation for the user, the representation generated using a Gaussian process.

22. The non-transitory machine-readable medium of claim 19, wherein the future activity comprises a workout, wherein the activity information comprises workout parameters, and wherein the physiological prediction comprises at least one of: a predicted heartrate zone for the user during the workout, a predicted heartrate for the user during the workout, a predicted number of calories that will be burned by the user during the workout, and a prediction of a potential cardiovascular event for the user during the workout.
